(19) United States
(12) Patent Application Publication
(10) Pub. No.: US 2002/0082688 A1
(43) Pub. Date: Jun. 27, 2002

(54) Technique for shrinking xenograft and allograft heart and vascular tissue

(76) Inventors: Tom Sander, Alachua, FL (US); Thien Doan, Alachua, FL (US); Tom Brewer, Alachua, FL (US)

Correspondence Address: VAN DYKE & ASSOCIATES, P.A., 1630 HILLCREST STREET, ORLANDO, FL, 32803, US

(21) Appl. No.: 09/962,924

(22) Filed: Sep. 25, 2001

Related U.S. Application Data
(60) Provisional application No. 60/235,776, filed on Sep. 25, 2000.

(51) Int. Cl. A61F 2/24

(52) U.S. Cl. 623/2.13; 623/918

(57) ABSTRACT

A process of applying thermal energy using a controlled regimen is described to reduce the size of biological xenograft and allograft heart and vascular tissue, particularly heart valves, which are used for implants by applying a controlled regimen of thermal energy, and the related implant products, apparatuses and systems. In a particular embodiment, a xenograft or allograft heart valve experiences size reduction and fits the size requirements of a recipient into whom the heart valve is being implanted.

### FIELD OF INVENTION

This invention relates to the process of reducing the size of biological xenograft and allograft heart and vascular tissue, particularly heart valves, which are used for implants by applying a controlled regimen of thermal energy, and the related implant products, apparatuses and systems related to reducing the size of xenograft and allograft heart tissue, vascular tissue, and heart valve implants.

### BACKGROUND OF THE INVENTION

A need exists in the medical arts to provide a sufficient quantity of suitable small-sized heart valves, largely for pediatric use. The valves from allograft organs often are too large for pediatric and female recipients, resulting in an imbalance in supply and demand for small heart valve implants. Alternatives for patients needing a small heart valve include constructing artificial heart valves, constructing heart valves from non-heart tissue, carving or cutting heart valves down to smaller sizes, or using porcine heart valves.

All of these alternatives have inherent difficulties. For instance, insertion of an artificial heart valve requires the recipient to take anticoagulation medication for the remainder of his or her lifetime. In contrast, a tissue, or biological heart valve replacement will require initial anticoagulation treatment, generally only for the postoperative period. Carving or cutting heart valves to size results in tissue damage to the valve, and risks improper functioning in the recipient. Use of a porcine heart valve will require a lifetime of taking immunosuppressant medication, and comes with the risk of contracting unknown or non-detected porcine disease agents.

Therefore a need exists for a reliable method to reduce the size of heart valves that are obtained from organ donors. A broader need exists for the ability to reduce the size of other heart and vascular xenograft and allograft tissue to be used for implant purposes. Currently, there is no recognized reliable technique to reduce the size of heart valves that are larger than will work for pediatric and other small-valve applications. The subject invention fills the need to supply heart valves for patients requiring valves that are smaller than donor hearts. The subject invention uses sources of thermal energy, such as radio frequency (RF) energy, to heat heart valve tissue in such a manner as to shrink the size of the heart valve while maintaining its utility for a transplant purpose. In this way large heart valves are shrunk in such a way that they can be used in smaller hearts. The same method is applied to the size reduction of heart and vascular xenograft and allograft tissue to be used for implant purposes.

Previous work in the general field of shrinking collagen and collagen-containing biological tissue deals to a large extent with eye tissues, joint tissues, and skin tissues, and employs treatments for use in vivo in a living patient. For example, U.S. Pat. No. 4,381,007 teaches the use of multipolar electrodes that emit RF energy to shape corneas in vivo by heating the collagen in the cornea. This patent notes the limitations of heating regimens that may damage adjacent tissues. U.S. Pat. No. 5,964,749 teaches the use of pulsed light to heat the skin, thereby shrinking collagen, while the surface is cooled with a transparent coolant. U.S. Pat. No. 6,036,687 teaches the use of an intravenous catheter that emits RF energy to shrink veins. U.S. Pat. No. 5,458,596 teaches the use of emitting RF energy through an electrode to shrink collagen in tissues, such as in ligaments, joint capsules, and connective tissue, as part of in vivo orthopedic treatments. U.S. Pat. No. 6,081,749 teaches an in vivo approach of applying energy to heat fascia and other support tissues to treat incontinence.

These and other references teach in vivo applications for specific autologous non-heart tissues. In addition, U.S. Pat. No. 5,928,224 teaches an apparatus that uses heat, pressure, or both, to reshape any misshapen portion of a heart valve during an in vivo procedure. U.S. Pat. No. 5,989,284 teaches the use of heat in a device and method to shorten the fibrous structures, chordae tendineae, that connect atrioventricular heart valves with papillary muscles. The invention is directed to resolving a valve prolapse condition, and is for an in vivo application. Also, U.S. Pat. No. 6,071,303 teaches heating myocardial infarct scar tissue with heat to reduce the scar tissue area. This again is for an in vivo application. These patents demonstrate the use of heat to specific areas of heart tissue structures for specific in vivo applications to treat specific disease states. These patents provide disclosure of value to one wishing to practice the present invention, and to that extent these references are hereby incorporated by reference. These patents neither teach nor suggest application of thermal energy to the whole heart valve for the implant purpose of the present invention. As such, the present invention represents an advance in the field that achieves the overall size reduction in heart valves used for implant purposes.

### SUMMARY OF THE INVENTION

The subject invention generally is directed to methods, implant products, apparatuses and systems related to reducing the size of xenograft and allograft heart valve implants, and heart and vascular xenograft and allograft tissue to be used for implant purposes. In particular, the subject invention is directed to the application of a controlled regimen of thermal energy to a heart valve, where the regimen of thermal energy results in shrinking the heart valve size while maintaining its required functionality for implant use. Thermal energy may be from a radio frequency energy generator or probe, or from other sources, such as but not limited to pulsed light, infrared radiation, ultrasonic energy, non-ablative laser energy, and thermal transfer. The thermal energy regimen also is applied to other heart and vascular xenograft and allograft tissue to be used for implant purposes.

It has been generally recognized in the art that heating will shorten collagen fibers, and that properly heated collagen-containing tissues will contract. It has been reported that temperatures between 43 to 90 degrees, 43 to 75 degrees, 45 to 60 degrees (most preferred of the three preceding ranges, in U.S. Pat. No. 5,569,242), 60 to 75 degrees, and up to 110 degrees (pulsed, when the target tissue is cooled, in U.S. Pat. No. 6,081,749) Celsius serve to shrink collagenous tissue. Critical to the success of the present method is the proper application of thermal energy to achieve such temperatures in the heart valve tissue, such that the required shrinkage is achieved without damage to the functionality and viability of the heart valve tissue. Often this involves specific regimens of heating, which can include surface cooling of tissue near the heat source, and specific types of sources of thermal energy.

The structure of the heart valve, and the need to maintain performance functionality of the implanted valve, require an appropriate controlled regimen of heating. The heart valve consists of two or three cusps, or leaflets, that contact each other in the closed position. When the chamber fills and the heart muscle for the chamber behind that valve contracts, the cusps open and blood flows through the opening. The aortic cusp, having an approximate thickness of 300 to 700 um, has three distinct layers, each containing different amounts of collagen. The top layer, facing the aorta, is called the fibrosa layer. It comprises about 45 percent of the valve thickness, and is the primary structural layer. The fibrosa layer contains a high quantity of collagen organized in bundles and fibers oriented circumferentially. A matrix of elastin, which is less stiff than collagen, surrounds the collagen bundles, helping to maintain microstructure during unloading.

The middle layer, the spongiosa, comprises about 25 percent of the valve thickness. It contains small amounts of collagen and elastin, which connect the fibosa layer to the third layer, called the ventricularis layer. Water is the major component of the spongiosa layer; glycosaminoglycans also are present.

The third layer, the ventricularis, is oriented toward the ventricle. It contains significant amounts of collagen and elastin, which are less directionally oriented than those in the fibrosa layer. Consequently, this layer is less stiff.

Thus, the application of thermal energy to the heart valve is directed to the balanced heating of the different heart valve layers to achieve a relatively uniform shrinking which preserves the integrity, functionality and viability of the valve for use as an implant. Therefore, in one embodiment, a heart valve is carefully surgically excised from a donor to retain all necessary structural components, which may include one or more of the associated chordae tendineae, papillary muscles, and valve annulus. The tissue is maintained sterile and cool during transport to the site of processing. Then an applicator applies a controlled regimen of radio frequency energy to heat the respective components of the valve to obtain a reduced size heart valve. The valve then is provided to the recipient. Optionally, final sizing by use of thermal energy may transpire during or after attachment of the valve implant into the recipient.

In essence, in one basic embodiment, the method of operation of the invention comprises:

1. Obtaining a heart valve from a xenograft or allograft heart that is sufficiently viable for use as a transplant to a recipient; and

2. Treating the heart valve prior to transplanting to the recipient with a regimen of thermal energy, whereby the regimen results in shrinkage of specific size parameters of the heart valve, resulting in an appropriately sized heart valve for the recipient.

What is claimed is:

1) A method of reducing the size of xenograft and allograft heart and vascular tissue comprising:
a. obtaining heart or vascular tissue from a xenograft or allograft source, or a combination thereof; and
b. treating the xenograft or allograft heart or vascular tissue prior to transplanting into a recipient in need thereof with a regimen of thermal energy,
whereby the regimen results in shrinkage of specific size parameters of the xenograft or allograft heart or vascular tissue, resulting in an appropriately sized xenograft or allograft heart or vascular tissue implant for the recipient.

2) A size-reduced xenograft or allograft heart or vascular tissue made by the process of claim 1.

3) A method of reducing the size of xenograft and allograft heart valves comprising:
a. obtaining a heart valve from a xenograft or allograft source, or a combination thereof; and
b. treating the heart valve prior to transplanting into a recipient in need thereof with a regimen of thermal energy,
whereby the regimen results in shrinkage of specific size parameters of the heart valve, resulting in an appropriately sized heart valve for the recipient.

4) A size-reduced heart valve made by the process of claim 3.

5) The method according to claim 3 comprising the additional step of storing the heart valve obtained from the xenograft or allograft source, or combination thereof, for a period of time prior to treating the heart valve with thermal energy.

6) The method according to claim 3 comprising the additional step of storing the heart valve obtained from the xenograft or allograft source, or combination thereof, for a period of time after treating the heart valve with thermal energy.

7) The method according to claim 3 comprising the additional step of transplanting the treated heart valve into the recipient.

8) The method according to claim 3 comprising the additional step of applying thermal energy to resize portions of the heart valve during transplantation of the treated heart valve into the recipient.

9) The method according to claim 3 comprising the additional step of selectively cooling portions of the heart valve during application of the thermal energy.

10) A method of reducing the size of xenograft and allograft heart valve tissue comprising:
a. obtaining a heart valve from a xenograft or allograft source, or a combination thereof; and
b. treating the heart valve prior to and substantially during transplantation into a recipient in need thereof with a regimen of thermal energy,
whereby the regimen results in shrinkage of specific size parameters of the heart valve, resulting in an appropriately sized heart valve for the recipient.

11) A method of reducing the size of xenograft and allograft heart valve tissue comprising:
a. obtaining a heart valve from a xenograft or allograft source, or a combination thereof; and
b. treating the heart valve during transplantation into a recipient in need thereof with a regimen of thermal energy,
whereby the regimen results in shrinkage of specific size parameters of the heart valve, resulting in an appropriately sized heart valve for the recipient.

12) A method of training a person to operate the thermal energy application device to reduce the size of heart valves and other collagen-containing xenograft and allograft tissue, or combination thereof, comprising preliminary instruction on the theory of thermal shrinking of collagen fibers in tissues; instruction on measurement of changes in size during use of the thermal energy application device, supervised practice using non-human tissues, and assistance to trained technicians for actual thermal applications to human tissue.

13) A method of treating a heart condition in a patient wherein said patient has a damaged, diseased, or defective heart valve, said method comprising the steps of:
a. removing said damaged, diseased, or defective heart valve; and,
b. implanting a xenograft or allograft heart valve in said patient
wherein said xenograft or allograft heart valve has been reduced in size by thermal energy.
